(19) United States
(12) Patent Application Publication
(10) Pub. No.: US 2002/0160067 A1
(43) Pub. Date: Oct. 31, 2002

(54) Therapeutic preparation and method for producing a therapeutic preparation using coffee beans as a substrate

(75) Inventors: Loretta M. Zapp, Austin, TX (US); Alan Andrews, Austin, TX (US); Lyndon C. Johnson, Austin, TX (US); Brian J. Slaga, Austin, TX (US)

Correspondence Address: DAVID G. HENRY, 700 Texas Center, P.O. Box 1470, Waco, TX, 76703-1470, US

(73) Assignee: Oncology Science Corporation, Austin, TX

(21) Appl. No.: 09/843,543

(22) Filed: Apr. 25, 2001

(51) Int. Cl. A61K 35/78; A23F 5/00; A23L 1/28

(52) U.S. Cl. 424/776; 426/432

(57) ABSTRACT

A raw green coffee bean extraction method and coffee extract end product. The method produces a coffee extract end product which contains antioxidants (phenolic compounds), diterpenes (having detoxification properties), has greater bioavailability and greater ability to quench oxidative stress in comparison to existing polyphenol extracts.

### BACKGROUND OF THE INVENTION

1. Field of The Invention

The present invention relates to dietary supplements and other therapeutic preparations, and to method for processing and producing such preparations. The instant invention is of extracts from coffee beans, which extracts are beneficial, in part, as anti-oxidants and anti-tumor agents.

2. Background Information

Phenolic compounds are known to be antioxidants and anti-tumor agents. The phenolic acids of chlorogenic acid, caffeic acid, para-coumaric acid and eugenol have been shown to exert cancer preventive activities in animal models.

Chlorogenic acid, an ester of caffeic acid and quinic acid, is an antioxidant in vitro and is suspected to be beneficial in preventing cardiovascular disease. Also, chlorogenic acid has shown to have a chemopreventative effect on rat stomach cancer, and to inhibit methylazoxymethanol-induced large intestinal tumors in hamsters.

In vivo assays show that one third of chlorogenic acid and almost all caffeic acid is absorbed in the small intestine of humans. This implies that part of the chlorogenic acid from foods will enter into the blood circulation, but most will reach the colon. Caffeic seems to be more bioavailable. (Olthof et al J Nutr 2001 January; 131 (1) :66-71).

Coffee contains phenolic acids which are mainly esters of quinic acid with different amount of caf feyl groups attached to its different positions.

Chlorogenic acid, which is the main phenolic acid in coffee, is able to protect the gastric mucosa against irritations, and, therefore, improves the digestibility of foods, beverages and medicaments. The improved digestibility is expressed through a much reduced systemic acid secretion (such as causes heartburn, etc.) which has been found to be directly dependent on an increased level of chlorogenic acid content in roasted coffee.

Despite the presence of phenolic compounds in coffee beans, and their known beneficial properties, phenolic compounds are conventionally obtained as extracts from green tea. This appears to be because coffee bean roasting processes reduce phenolic content in coffee beans between 40% and 80%, and no one has heretofore considered obtaining phenolic compounds from green coffee beans. Analysis by the present inventors indicate that green coffee beans which initially contain 4% phenolic acids contain, respectively, 2% phenolic acids when light roasted, 1% when medium roasted, and less than 0.5% when dark roasted. This clearly represents a significant loss of beneficial compounds through the roasting process.

Not only are green coffee beans a source of beneficial phenolic compounds, but work by the present inventors indicate that, unit weight per unit weight, green coffee beans produce more beneficial phenolic compounds, and in a more beneficial constituency than that obtained in the form of extracts from green tea.

It will be of benefit to humankind to provide any new and therapeutic preparation which has anti-oxidant and anti-tumor properties. It will likewise be of benefit to provide improved such preparations and/or new and more prolific methods for obtaining or producing such preparations.

### SUMMARY OF THE INVENTION

In view of the foregoing, it is an object of the present invention to provide a beneficial preparation with healthful benefits.

It is another object of the present invention to provide a preparation with constituents which are believed to have beneficial antioxidant properties when consumed by humans.

It is another object of the present invention to provide a preparation with constituents which are believed to have beneficial anti-tumor properties when consumed by humans.

It is another object of the present invention to provide a health-promoting preparation with constituents which, while obtainable from alternative sources, are present in more beneficial quantities or ratios than are presently available through conventional source materials or through practice of conventional processes.

It is another object of the present invention to provide a new method for producing phenolic compound rich preparations.

It is another object of the present invention to provide a new method for producing more phenolic compound rich preparations than conventional methods produce.

It is another object of the present invention to provide a new method for producing phenolic compound rich preparations with constituents which, while obtainable from alternative sources, are present in more beneficial quantities or ratios than are presently available through conventional source materials or through practice of conventional processes.

It is another object of the present invention to provide a new method for producing phenolic compound rich preparations from source materials not heretofore recognized as a practical source of such compounds.

It is another object of the present invention to provide a new method for extracting phenolic compounds from coffee beans.

It is another object of the present invention to provide a novel, extract, which may be used as a dietary supplement, flavoring, or functional food containing an extract of green coffee beans which contains polyphenolic acids and other beneficial compounds, such as diterpenes.

It is another object of the present invention to provide an improved extract processing method which yields an extract of polyphenolic acids and other beneficial compounds which are healthier than existing polyphenol extracts.

It is another object of the present invention to provide an improved extract which is more bioavailable than polyphenol extracts which are processed by conventional methods.

It is another object of the present invention to provide an improved extract which has greater ability to quench oxidative stress and destroy free radicals than polyphenol extracts which are processed by conventional methods.

It is another object of the present invention to provide an improved raw green coffee bean extract which yields a more healthful end product than existing polyphenol extracts.

In satisfaction of these and related objects, the present invention is of extracts from coffee beans, which extracts contain beneficial measures of phenolic acids, as well as of processes for producing such extracts. Remarkably, the simple method taught herein produces an extract product which is more bioavailable, contains a healthier profile of antioxidants (phenolic compounds) and more diterpenes (having detoxification properties) than any existing phenolic compound-focused product.

### DETAILED DESCRIPTION OF THE PREFERRED EMBODIMENT

The phenolic compound rich extract which is the focus of the present invention is produced from raw green coffee beans. This is a significant departure from conventional methods for producing polyphenol focused products, where green tea is the source material. Not only is the source for phenolic compounds different in this case, the end product of the present invention exhibits a chemical profile of phenolic acids and other beneficial compounds which is more beneficial than the existing, tea-based polyphenol extracts. The new process of the present invention yields more active, more bioavailable, and larger quantities (per unit weight of source material) of phenolic compounds than may be achieved through conventional extracting methods, and are found in existing phenolic compound-focused, green tea-based extracts.

### EXAMPLE

Raw green beans of any origin are ground in a commercial grinder to a very fine powder. (less than 0.63 mm particles). The grinding of the beans to a fine consistency increases the surface area of each particle which, in turn, enhances the extraction process by increasing yield of phenolic compounds and other beneficial compounds. According to certain established analytical standards, which may well be used as an appropriate guide for these purposes, green coffee beans should be ground to pass through a number 40 sieve. In any event, the ground coffee should be kept rather cold (30° F., for example) to avoid pastiness.

The ground coffee powder is then extracted with an alcohol (methanol, hexane, other alcohol) water solution. The presently believed preferred mode is based on a methanol/water 60% mixture.

Conventionally, green tea is extracted with ethanol/water mixture, and the same will likely perform acceptably for the present method. The beans may also be extracted by use of a Super Critical Fluid extraction process, which the present inventors believe may produce/extract antioxidants that would not otherwise be produced through extracting green coffee beans, or would not be produced in as great a comparative volume per unit source material.

Whatever the extracting agent, the beans may be extracted more than once to enhance the process and obtain greater yields of phenolic compounds.

Total chlorogenic acid content of green arabica beans is typically 6.9%. Robusta is typically 10%. A number of different chlorogenic acids are present—5-caffeoylquinic acid is present in the largest amount. Dicaffeoyl and feruloyl quinnic acids are also present together with the 3 and 4-isomers of monocaffeoylquinic acid.

Green coffee beans typically contain 1.3% diterpenes in arabica and 0.2% diterpenes in green robusta. The diterpenes are cafestol and kahweol. Various sterols and tocopherols are also present in the lipid part of green beans. The presence of alkanoylated 5-hydroxytryptamines (the Japanese compound) in the wax on the outer surface of the green beans are present at 500-1000 mg/kg.

Trigonelline is present at 1.1% is arabica and 0.65% in robusta. Trigonelline is transformed somewhat into nicotinic acid.

Apart from chlorogenic acids, the main acids present in significant quantities are quinic, malic, citric, lactic, pyruvic, succinic and glycolic.

The extract may also be derived from ground roasted beans, or brewed coffee extracts derived from raw beans, roasted beans of varying degree (the less roasted—the more polypohenols remain), or derived from brewed coffee, but the yields of phenolic compounds will not be as great as if produced from green coffee beans, for reasons explained above.

Once produced, the extracts of the present invention are wholly or partially dehydrated and packaged as oral dosage forms in typical dietary supplement format, added to foods, and/or delivered in a medium for topical, cosmetic use (such as in a cream or ointment, for example). If the extracts are to be consumed directly (as a food additive, for example), they may be flavored, and thereby serve as a dual-purpose product (as a coffee-flavoring agent, for example).

In the preceding examples, all percentages are reported by weight. The chlorogenic acid [list other compounds] contents given were obtained by high-pressure liquid chromatography (HPLC) and UV photometric methods.

The Linoleic Acid Autoxidation method is a rapid screening test to determine antioxidant potencies of natural and synthetic antioxidants. It measures antioxidant efficiencies in a model system consisting of micelles of sodium dodecyl sulfate (SDS) with added linoleic acid. The method involves following the development of absorption at 234 nm which results when linoleic acid is oxidized to its conjugated diene hydroperoxide by the addition of the compound ABAP (2,2′-azobis(2-amidinopropane) dihydrochloride), the initiator. The antioxidant efficiency (the rate constant for the reaction of the peroxyl radical from linoleic acid with the antioxidant divided by the propagation rate constant for autoxidation of linoleic acid) of the test substance is compared to that of alpha tocopherol. Then absolute value of the rate constant for the reaction of alpha tocopherol with peroxyl radicals is known in micellar systems.

The preceding examples illustrate that a more healthful polyphenol extract product can be produced by a very simple variation of conventional polyphenol extraction methods. In addition, an end product which is healthier and cheaper than existing polyphenol extracts can be produced, and thereby provide an economic benefit to vendors. The present method yields a product which is in no way undesirable from an aesthetic standpoint. Thus, there is no reason not to, and every reason to, adopt the present coffee extraction processing methods for the well being of consumers.

Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

I claim:

1. A method for producing a phenolic compound rich preparation comprising the steps of:
selecting a measure of coffee beans;
grinding said coffee beans to form a powder; and
applying an extracting agent to said powder for extracting phenolic compounds from said powder.

2. The method of claim 1 wherein said coffee beans are substantially raw, green coffee beans.

3. The method of claim 1 wherein said extracting agent is comprising water and an alcohol.

4. The method of claim 2 wherein said extracting agent is comprising water and an alcohol.

5. The preparation of claim 2 produced through the steps thereof.
